(19) United States
(12) Patent Application Publication
(10) Pub. No.: US 2022/0291180 A1
(43) Pub. Date: Sep. 15, 2022

(54) FURAN CONCENTRATION QUANTIFYING METHOD, TRANSFORMER DETERIORATION DIAGNOSIS METHOD USING SAME, AND APPARATUS THEREFOR

(71) Applicant: KOREA ELECTRIC POWER CORPORATION, Naju-si (KR)

(72) Inventors: Hyun-Joo PARK, Daejeon (KR); Byeong-Sub KWAK, Daejeon (KR); Beom-Joo KIM, Daejeon (KR); Ah-Reum KIM, Daejeon (KR); Tae-Hyun JUN, Daejeon (KR)

(21) Appl. No.: 17/636,849

(22) PCT Filed: Jun. 10, 2020
(86) PCT No.: PCT/KR2020/007489

§ 371 Date: Feb. 18, 2022

(30) Foreign Application Priority Data
Aug. 21, 2019 (KR) 10-2019-0102500

(51) Int. Cl. G01N 30/74; G01N 33/28

(52) U.S. Cl. CPC G01N 30/74; G01N 33/2835; G01N 33/2888; G01N 2030/027

(57) ABSTRACT

Disclosed is a method of quantifying furan concentration to efficiently manage a deterioration state of a transformer in the field. The method of quantifying furan concentration includes: measuring a color-development degree of an extraction solution by making the extraction solution containing furan extracted from an insulating-oil sample mix and react with a color reagent; and quantifying furan concentration by correction based on a correlation between a precise analysis and a simple analysis with regard to the color-development degree of the extraction solution, wherein the precise analysis is to obtain a quantitative value through color column separation based on high performance liquid chromatography (HPLC) in a laboratory to analyze a furan compound in insulating oil of a transformer, and the simple analysis is to obtain a chromaticity value in a field with regard to actual transformer samples.

### TECHNICAL FIELD

The disclosure relates to a method of quantifying furan concentration, and a method and apparatus for diagnosing transformer deterioration using the same, and more particularly to a method of quantifying furan concentration and a method and apparatus for diagnosing transformer deterioration using the same, in which furan concentration of an insulating-oil sample taken from a transformer is quantified, and the quantified furan concentration is used to diagnose a deterioration state of the transformer to thereby efficiently manage the deterioration state of the transformer in the field.

### BACKGROUND ART

In general, the life of a transformer is restricted by aging or deterioration of insulating paper wound around a winding of the transformer. However, it is virtually impossible to directly look into the transformer, and therefore an indirect method of evaluating the insulating paper inside the transformer is needed.

The insulating paper is made of cellulose, and cellulose mainly contains glucose molecular units and thus produces a pentagonal furan-based compound by a condensation reaction.

Insulating oil begins to thermally decomposed at its boiling point of 400° C. or higher, and increases in this thermal decomposition as temperature rises, thereby generating deterioration products such as a furan-based compound, i.e., furfural(2-furaldehyde), etc. At this time, CH₄, C₂H₆, C₂H₄, and the like low molecular hydrocarbon gases and hydrogen gas (H₂), which are highly soluble in the insulating oil, are mostly generated.

Meanwhile, high performance liquid chromatography (HPLC) is generally used to analyze the furan compound in the insulating oil of the transformer. Furan is primarily extracted from the oil and then analyzed by the HPLC with a flowing mobile phase solvent. Such an analysis method is a classical organic compound analysis method that needs expensive equipment and skilled professionals. Therefore, it takes much time and maintenance is difficult. In other words, this analysis method cannot make a direct diagnosis in the field.

Accordingly, there has been proposed a furan diagnosis apparatus that can be used even by nonprofessionals in the field because it employs an analysis method based on a chemical reaction between the furan compound and aniline-acetate. Such a furan diagnosis apparatus can quantify furan concentration based on a color-development degree of a pink complex generated by a color reaction between the furan compound and aniline-acetate, and diagnose deterioration of a decrepit transformer.

However, a simple analysis method based on the color reaction is to observe chromaticity with the naked eyes or is used as a method of screening at certain concentration or higher. This qualitative analysis method cannot replace a quantitative analysis method as compared to a laboratory analysis method.

In the case of using the existing commercial colorimeter, quantification is possible after correction using a standard substance, but there are disadvantages of having to take data to the outside and use a quantification program when a correction program is not installable according to manufacturers.

Further, a nonprofessional cannot know what the quantified furan concentration means even though they s/he uses the furan diagnosis apparatus.

Therefore, the existing furan diagnosis apparatus requires an algorithm for selecting and correcting an optimal color wavelength to accurately measure the concentration.

### DISCLOSURE

### Technical Problem

The disclosure is to provide a method of quantifying furan concentration and a method and apparatus for diagnosing transformer deterioration using the same, in which furan concentration of an insulating-oil sample taken from a transformer is quantified, and the quantified furan concentration is used to diagnose a deterioration state of the transformer to thereby efficiently manage the deterioration state of the transformer in the field.

### Technical Solution

According to an embodiment of the disclosure, a method of quantifying furan concentration includes: measuring a color-development degree of an extraction solution by making the extraction solution containing furan extracted from an insulating-oil sample mix and react with a color reagent; and quantifying furan concentration by correction based on a correlation between a precise analysis and a simple analysis with regard to the color-development degree of the extraction solution, wherein the precise analysis is to obtain a quantitative value through color column separation based on high performance liquid chromatography (HPLC) in a laboratory to analyze a furan compound in insulating oil of a transformer, and the simple analysis is to obtain a chromaticity value in a field with regard to actual transformer samples.

The extraction solution may be separated as a layer in a lower side as the insulating-oil sample and the extraction solvent are mixed and settled in a vertical direction.

The insulating-oil sample and the extraction solvent may be mixed and shaken side to side at 90 degrees for 1 minute or shaken 50 times by a sample mixer.

A ratio of aniline to acetate may be 1:6 for the color reagent.

A ratio of the extraction solution and the color reagent may be 1:1.5, and a reaction between the extraction solution and the color reagent may be carried out for 4 minutes.

The measuring the color-development degree of the extraction solution may include measuring the color-development degree of the extraction solution in a wavelength range of 520 nm and 530 nm.

The correlation between the precise analysis and the simple analysis may include a reliable level verified through a Pearson correlation analysis based on a Pearson correlation coefficient (r) calculated by the following equation:

$$r = \frac{{S}_{xy}}{\sqrt{{S}_{xx} {S}_{yy}}} ( where , {S}_{xx} = {\sum }_{i = 1}^{n} {x}_{i}^{2} - \frac{{( {\sum }_{i = 1}^{n} {x}_{i} )}^{2}}{n} , {S}_{yy} = {\sum }_{i = 1}^{n} {y}_{i}^{2} - \frac{{( {\sum }_{i = 1}^{n} {y}_{i} )}^{2}}{n} , {S}_{xy} = {\sum }_{i = 1}^{n} {x}_{i} {y}_{i} - \frac{( {\sum }_{i = 1}^{n} {x}_{i} ) ( {\sum }_{i = 1}^{n} {y}_{i} )}{n} , \qquad [ Equation ]$$

and there is a linear relationship between two variables x and y).

According to an embodiment of the disclosure, a method of diagnosing transformer deterioration includes: quantifying furan concentration of an insulating-oil sample taken from a transformer; and diagnosing a deterioration state of the transformer through the quantified furan concentration based on a correlation between the furan concentration and a degree of polymerization of insulating paper.

The quantifying may include: measuring a color-development degree of an extraction solution by making the extraction solution containing furan extracted from an insulating-oil sample mix and react with a color reagent; and quantifying furan concentration by correction based on a correlation between a precise analysis and a simple analysis with regard to the color-development degree of the extraction solution, wherein the precise analysis is to obtain a quantitative value through color column separation based on high performance liquid chromatography (HPLC) in a laboratory to analyze a furan compound in insulating oil of a transformer, and the simple analysis is to obtain a chromaticity value in a field with regard to actual transformer samples.

The diagnosing may include diagnosing the deterioration state of the transformer with respect to furan concentration through a Chendong model showing a correlation between the furan concentration and the degree of polymerization of the insulating paper.

The diagnosing may include providing stepwise diagnosis results with respect to furan concentration at which the degree of polymerization of the insulating paper is lowered to 50% or less.

The diagnosis results may be providable by each of a distribution transformer and a transmission and transformation transformer.

According to an embodiment of the disclosure, an apparatus for diagnosing transformer deterioration includes: at least one processor; and a memory configured to store computer readable instructions, wherein, the instructions are executed by the at least one processor allow the apparatus for diagnosing the transformer deterioration to: quantify furan concentration of an insulating-oil sample taken from a transformer; and diagnose a deterioration state of the transformer through the quantified furan concentration based on a correlation between the furan concentration and a degree of polymerization of insulating paper.

The instructions may be executed by the at least one processor allow the apparatus for diagnosing the transformer deterioration to: measure a color-development degree of an extraction solution by making the extraction solution containing furan extracted from an insulating-oil sample mix and react with a color reagent, when furan concentration is quantified; and quantify furan concentration by correction based on a correlation between a precise analysis and a simple analysis with regard to the color-development degree of the extraction solution, wherein the precise analysis is to obtain a quantitative value through color column separation based on high performance liquid chromatography (HPLC) in a laboratory to analyze a furan compound in insulating oil of a transformer, and the simple analysis is to obtain a chromaticity value in a field with regard to actual transformer samples.

The extraction solution may be separated as a layer in a lower side as the insulating-oil sample and the extraction solvent are mixed and settled in a vertical direction, and the apparatus may further include a sample mixer configured to mix and shake the insulating-oil sample and the extraction solvent side to side at 90 degrees for 1 minute or 50 times.

The apparatus may further include a display configured to display the quantified furan concentration, and stepwise diagnosis results with respect to the quantified furan concentration.

### Advantageous Effects

According to the disclosure, furan concentration of an insulating-oil sample taken from a transformer is quantified, and the quantified furan concentration is used to diagnose a deterioration state of the transformer, thereby efficiently managing the deterioration state of the transformer in the field.

Further, according to the disclosure, furan in insulating oil is quantified without qualitatively comparing colors with the naked eyes for each concentration class according to change in chromaticity, and diagnostic criteria are separately prepared for a transmission and transformation transformer and a distribution transformer according to voltage levels, thereby diagnosing the deterioration state.

Further, according to the disclosure, deterioration products are accurately quantified for integrity evaluation in consideration of life increase of a long-running transmission and distribution transformer, and a diagnostic algorithm for each voltage level is used, thereby finally diagnosing the deterioration state of the transformer.

Further, according to the disclosure, an algorithm for indirectly predicting a degree of polymerization of the insulating paper is included for deterioration evaluation of a decrepit transformer, thereby eliminating human-based high-uncertainty factors of lowering a diagnosis accuracy in a screening diagnosis method based on a chromaticity table with only chromaticity values.

Further, according to the disclosure, even nonprofessionals can check diagnosis results through an analysis improved in accuracy and a diagnostic algorithm capable of predicting a degree of polymerization.

Further, the disclosure is not only easily and quickly applicable in the field but also secures competitiveness in terms of diagnostic accuracy, thereby remarkably improving the management of the transformer.

Further, according to the disclosure, unit costs are reduced by about ⅕ as compared to those of a laboratory analysis to thereby increase the efficiency of on-site diagnosis, and an improved accuracy and a diagnostic algorithm are provided as compared to the precise analysis of the furan concentration in the laboratory.

### DESCRIPTION OF DRAWINGS

FIG. 1 shows a method of quantifying furan concentration according to an embodiment of the disclosure,

FIG. 2 shows color-development degrees according to concentrations of aniline acetate (1:3),

FIG. 3 shows absorbance values (1:3) per minute according to the concentrations of FIG. 2,

FIG. 4 shows color-development degrees according to concentrations of aniline acetate (1:6),

FIG. 5 shows absorbance values (1:6) per minute according to the concentrations of FIG. 4,

FIG. 6 shows reaction values of a standard substance per minute according to wavelengths for each concentration,

FIG. 7 shows reaction values of a standard substance according to concentrations at 520 nm,

FIG. 8 shows reaction values of a standard substance according to concentrations at 530 nm,

FIG. 9 shows a color-development photograph of 1 [symbol] of a reaction sample and 300 [symbol] of a color reagent (1:6),

FIG. 10 shows a color-development photograph of 1 [symbol] of a reaction sample and 1 [symbol] of a color reagent (1:6),

FIG. 11 shows absorbance values per minute (1 [symbol] of an extraction solution, 300 [symbol] of a color reagent) according to color-reagent concentrations,

FIG. 12 shows absorbance values per minute (1 [symbol] of an extraction solution, 1 [symbol] of a color reagent) according to color-reagent concentrations,

FIG. 13 shows absorbance values per minute (1.5 [symbol] of an extraction solution, 1 [symbol] of a color reagent) according to color reagent concentrations,

FIG. 14 shows result data of a precise analysis and a simple analysis,

FIG. 15 shows result comparison between the precise analysis and the simple analysis,

FIG. 16 shows Pearson correlation analysis results between precise and simple analysis results,

FIG. 17 shows a method of diagnosing transformer deterioration according to an embodiment of the disclosure,

FIG. 18 shows a deterioration mechanism of cellulose molecules,

FIG. 19 shows a furan-based compound,

FIG. 20 shows analysis results of a degree of polymerization of insulating paper in ground transformers,

FIG. 21 shows a relationship between furan concentration of an actual onsite sample and a degree of polymerization of the insulating paper in FIG. 20,

FIG. 22 shows a relationship between furan concentration and an average degree of polymerization of the insulating paper,

FIG. 23 shows furan diagnostic criteria for a distribution transformer,

FIG. 24 shows furan diagnostic criteria for a transmission and transformation transformer,

FIGS. 25 to 27 shows an apparatus for diagnosing transformer deterioration according to an embodiment of the disclosure.

### MODE FOR INVENTION

Hereinafter, exemplary embodiments of the disclosure will be described in detail with reference to the accompanying drawings. However, detailed descriptions about publicly known functions or features, which may obscure the gist of the disclosure in the following descriptions and the accompanying drawings, will be omitted. If possible, like numerals refer to like elements throughout the drawings.

Terms or words used in this specification and claims set forth herein should not be construed as being limited to conventional or lexical meaning, but be construed as meanings and concepts consistent with the technical spirit of the disclosure on the principle that the inventor can appropriately define the terms to explain his/her invention in the best way.

Therefore, the embodiments described in this specification and the configurations illustrated in the drawings are merely the exemplary embodiments of the disclosure and do not represent all the technical ideas of the disclosure, and thus it should be appreciated that there may be various equivalents and modifications at the filing date of the present application.

Below, an embodiment of the disclosure will be described in detail with reference to the accompanying drawings so as to be easily carried out by a person having ordinary knowledge in the art to which the disclosure pertains. However, the disclosure may be embodied in many different forms and not limited to the embodiments set forth herein. In the accompanying drawings, elements unrelated to the description are omitted for clarity, and like elements are denoted by like numerals throughout.

Below, exemplary embodiments of the disclosure will be described with reference to the accompanying drawings.

An apparatus for diagnosing transformer deterioration according to an embodiment of the disclosure quantifies furan concentration at the maximum color-development wavelength by extracting furan (furfural) from insulating oil of a transformer with an optimal extraction solvent, and then diagnoses the deterioration of the transformer with respect to furan concentration derived through a quantitative analysis by applying a diagnostic algorithm based on a correlation between the furan concentration and a degree of polymerization of the insulating paper according to voltage levels.

Like this, the apparatus for diagnosing the transformer deterioration does not perform a qualitative analysis by which an observer compares colors corresponding to concentrations with his/her naked eyes in consideration of variation in a color-development degree of furan in the insulating oil of the transformer, and is therefore capable of eliminating uncertainties caused by the observer.

In addition, the apparatus for diagnosing the transformer deterioration can accurately diagnose the deterioration of the transformer by preparing deterioration diagnostic criteria for a transmission and transformation transformer and a distribution transformer according to voltage levels based on furan concentration derived through a quantitative analysis.

Prior to description of such an apparatus for diagnosing the transformer deterioration, a method of quantitatively analyzing the furan concentration and a method of diagnosing the transformer deterioration, which are used by the apparatus for diagnosing the transformer deterioration, will be described in detail.

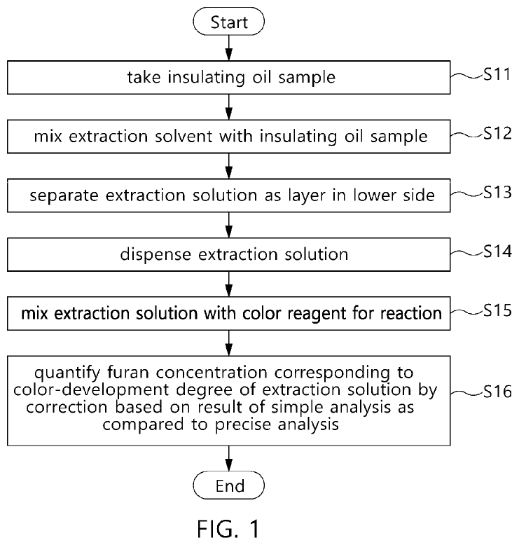

FIG. 1 shows a method of quantifying furan concentration according to an embodiment of the disclosure.

As shown in FIG. 1, an insulating-oil sample is taken from a transformer of which deterioration will be identified (S11). Here, 10 [symbol] of insulating-oil sample is taken.

Then, the insulating-oil sample and an extraction solvent are mixed (S12). In this case, the insulating-oil sample and the extraction solvent are injected into an econo-pac chromatography column (hereinafter, referred to as a ‘column’), and uniformly mixed by a sample mixer. Further, the sample mixer automatically shakes the column from side to side for one minute (about 50 times).

Here, the column is packed with 500 [symbol] (total volume) of predetermined resin, and a column lid is opened to inject the extraction solvent and the insulating-oil sample. Further, 3 [symbol] of 40% methanol is injected as the extraction solvent to extract furan, and 10 [symbol] of insulating-oil sample is injected.

Then, the mixed solution of the insulating-oil sample and the extraction solvent is settled, and the extraction solution is separated as a layer in a lower side (S13). In this case, the column lid is opened and the column is settled standing up in a vertical direction.

Then, the extraction solution with extracted furan is partially taken (S14). In this case, a lower tip of the column is removed, and 1.5 [symbol] of the extraction solution is dispensed into a vial.

Then, the extraction solution (i.e., a reaction sample) is mixed with and reacts with a color reagent (S15). In this case, a reaction time is given for 4 minutes.

Here, the color reagent refers to a chemical reagent in which a ratio of aniline to acetate is 1:6. Further, 1 [symbol] of the color reagent and 1.5 [symbol] of the extraction solution are used. That is, a ratio of the color reagent to the extraction solution is 1:1.5.

Then, furan concentration corresponding to a color-development degree of the extraction solution (i.e., furan concentration) is quantified by correction based on the result of a simple analysis as compared to a precise analysis (S16).

Meanwhile, to obtain an optimum wavelength range after the extraction solution reacts with the color reagent and turns pink, the ratio in the color reagent, and the reaction time and ratio of the extraction solution to the color reagent, which are mentioned in FIG. 1 are set as follows.

To this end, according to the disclosure, experiments were carried out in order described above in FIG. 1, and the ratio in the color reagent, and the ratio and reaction time of the extraction solution and the color reagent were set based on the experimental results. Herein, a new insulating-oil standard substance may be used instead of the insulating-oil sample.

First, to derive the optimum ratio in the color reagent of FIG. 1, the ratios of aniline and acetate, for example, 1:3 and 1:6 were compared.

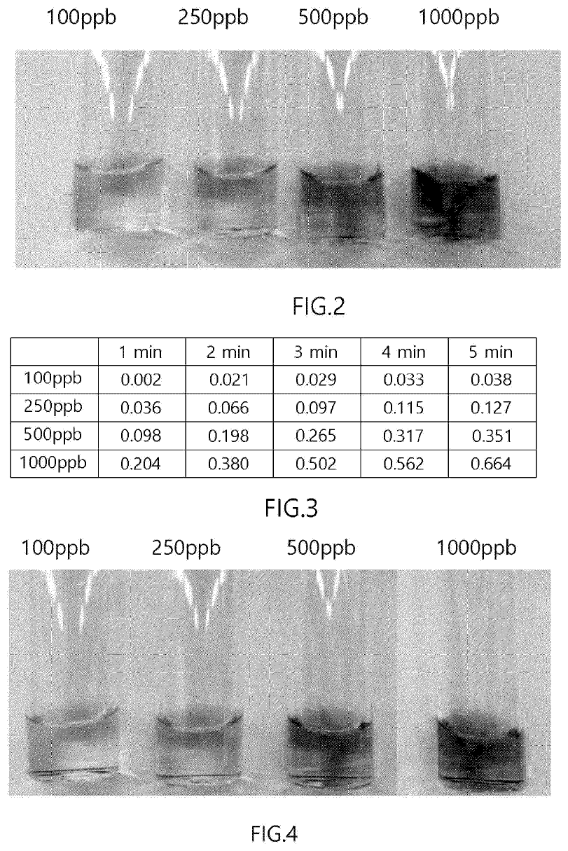

FIG. 2 shows color-development degrees according to concentrations of aniline acetate (1:3), FIG. 3 shows absorbance values (1:3) per minute according to the concentrations of FIG. 2, FIG. 4 shows color-development degrees according to concentrations of aniline acetate (1:6), and FIG. 5 shows absorbance values (1:6) per minute according to the concentrations of FIG. 4.

Referring to FIGS. 2 to 5, a chromaticity value was a little higher at the ratio of 1:3.

However, a high percentage of aniline causes the color reagent's own color to have an effect when the extraction solution turns color.

Therefore, the ratio of aniline to acetate in the color reagent is set at 1:6 by increasing the percentage of acetate to have an optimum chromaticity reaction. In other words, in the step S15 of FIG. 1, the color reagent in which the ratio of aniline to acetate is 1:6, is used.

Next, in a commercial colorimeter, the wavelength range in which the extraction solution reacts with the color reagent and turns pink is detectable at 520 nm and 530 nm.

To derive the optimum reaction time in FIG. 1, colorimeter reaction values were compared with respect to a color reaction per minute.

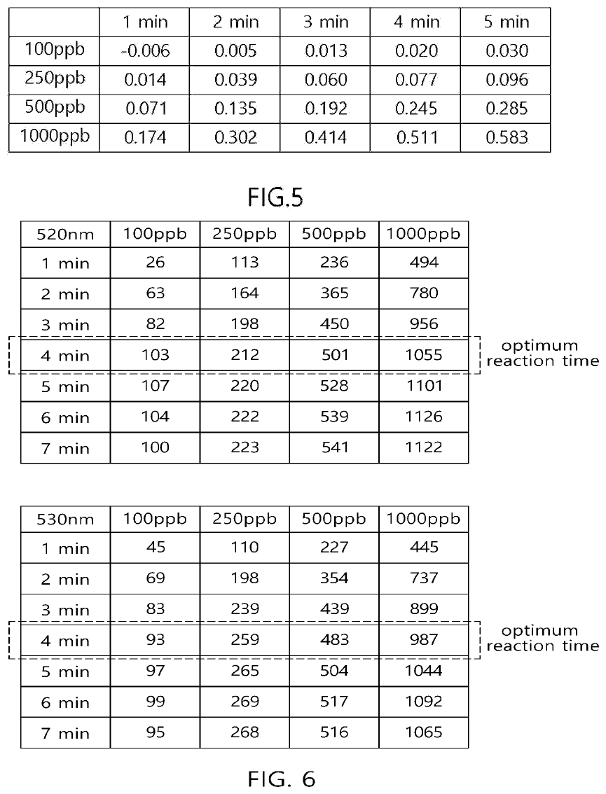

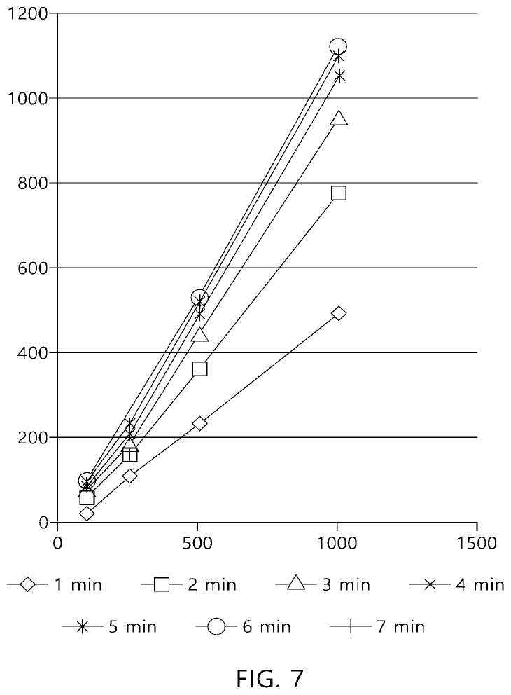

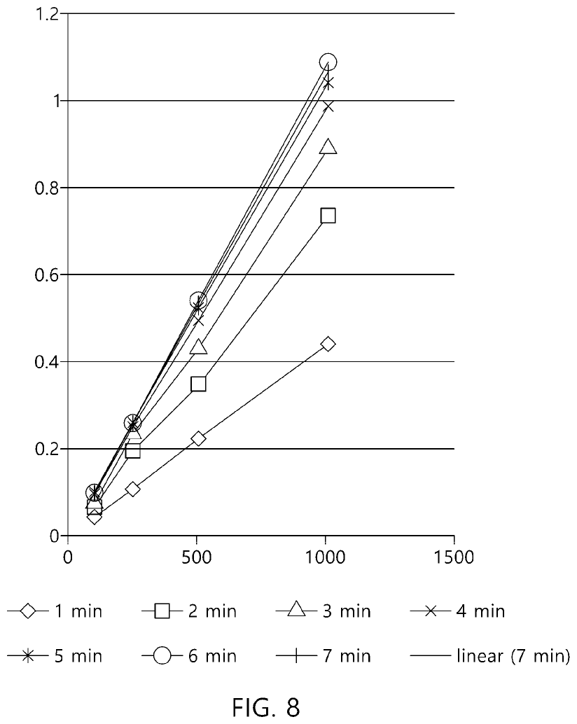

FIG. 6 shows reaction values of a standard substance per minute according to wavelengths for each concentration, FIG. 7 shows reaction values of a standard substance according to concentrations at 520 nm, and FIG. 8 shows reaction values of a standard substance according to concentrations at 530 nm.

Referring to FIGS. 6 to 8, there was no significant difference between the reaction values at 520 nm and 530 nm. Thus, the optimum wavelength region (wavelength range) from 520 nm to 530 nm may be selected.

To strengthen the advantages of an onsite diagnostic kit, it is not preferable that the reaction time is longer than 10 minutes. Thus, results within the reaction time of 7 minutes were reflected, and the optimum reaction time in FIG. 6 was set to 4 minutes.

The optimum reaction time corresponds to time taken in shaking side to side at 90 degrees about 50 times when furan is extracted by an automatic mixer. Such an optimum reaction time may be set to prevent furan from being excessively mixed to form an emulsion while extracting furan.

Next, to derive the ratio of the extraction solution to the color reagent in FIG. 1, 1 [symbol] and 1.5 [symbol] of the extraction solutions to react with the minimum of 300 [symbol] and the maximum of 1 ml of color reagent were compared.

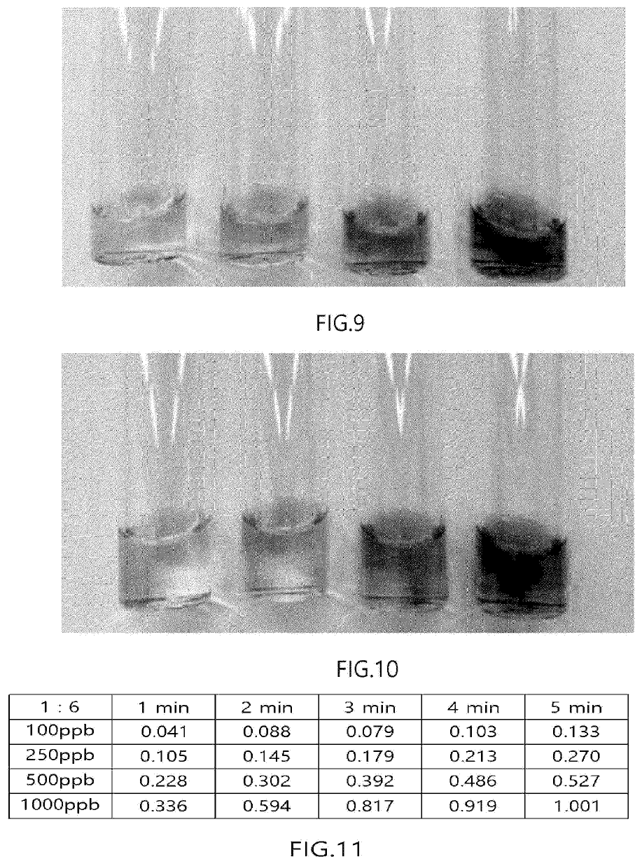

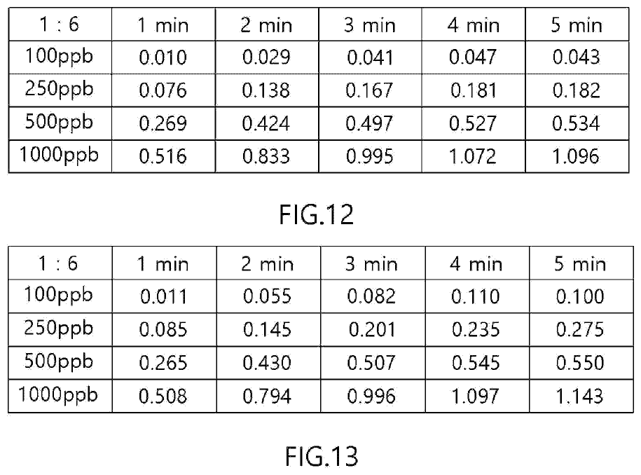

FIG. 9 shows a color-development photograph of 1 [symbol] of the reaction sample and 300 [symbol] of the color reagent (1:6), FIG. 10 shows a color-development photograph of 1 [symbol] of the reaction sample and 1 [symbol] of the color reagent (1:6), FIG. 11 shows absorbance values per minute (1 [symbol] of the extraction solution, 300 [symbol] of the color reagent) according to color-reagent concentrations, FIG. 12 shows absorbance values per minute (1 [symbol] of the extraction solution, 1 ml of the color reagent) according to color-reagent concentrations, and FIG. 13 shows absorbance values per minute (1.5 [symbol] of the extraction solution, 1 [symbol] of the color reagent) according to color reagent concentrations.

Referring to FIGS. 9 to 13, when the same amount of extraction solution is given, the more the color reagent is mixed, the darker pink is developed. Further, the chromaticity value is high when the maximum of 1.5 [symbol] of the extraction solution is used.

Accordingly, the ratio of the extraction solution to the color reagent was selected at 1.5:1. In other words, 1.5 [symbol] of the extraction solution and 1 [symbol] of the color reagent may be used.

Meanwhile, to quantify the furan concentration with respect to the color-development degree of the extraction solution in the step S16 of FIG. 1, a correlation of results between a precise analysis and a simple analysis was confirmed.

Here, the ‘precise analysis’ refers to obtainment of quantitative values through color column separation based on high performance liquid chromatography (HPLC) in a laboratory so as to analyze a furan compound in the insulating oil of the transformer, and the ‘simple analysis’ refers to obtainment of chromaticity values in the field with regard to actual transformer samples. In other words, the simple analysis refers to obtainment of the chromaticity value by the apparatus for diagnosing the transformer deterioration.

Thus, based on a correlation of results between the precise analysis and the simple analysis (i.e., results of the simple analysis as compared to the precise analysis), the result of the simple analysis may be quantified by correction up to a reliable level corresponding to the result of the precise analysis. In this way, the furan concentration obtained by the apparatus for diagnosing the transformer deterioration is quantified by correction up to a reliable level corresponding to the result of the precise analysis.

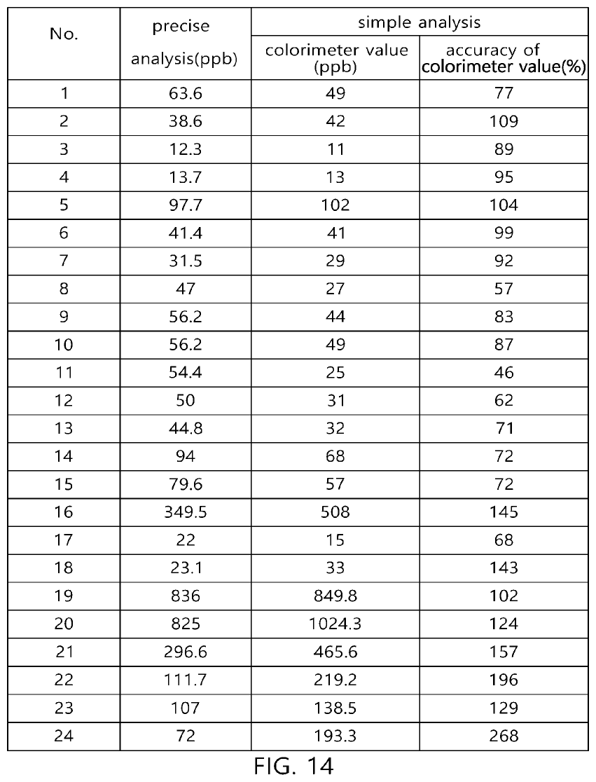

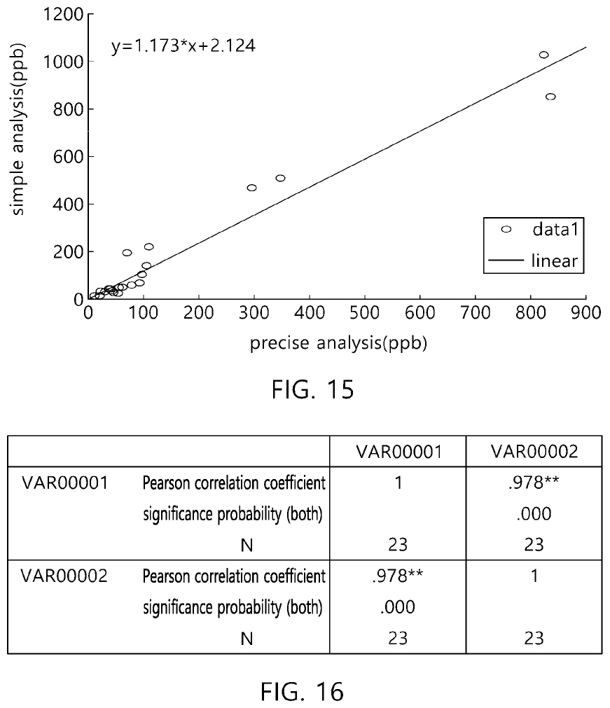

FIG. 14 shows result data of the precise analysis and the simple analysis, and FIG. 15 shows result comparison between the precise analysis and the simple analysis.

Referring to FIG. 14, the reliable level was checked by comparison between the results of the simple analysis, obtained by the apparatus for diagnosing the transformer deterioration, and the results of the precise analysis using the HPLC.

In addition, to eliminate human uncertainties, the automatic mixer was introduced and set considering that 4 minutes are taken in shaking side to side at 90 degrees about 50 times to extract furan.

Further, to increase the accuracy of comparison and verification, the samples to be compared are taken from actually operating distribution transformers in 24 fields.

Referring to FIG. 15, a graph of comparison in results between the precise analysis and the simple analysis result shows that result values of the simple analysis are higher by about 17% than those of the precise analysis. In this case, there was no significant difference between overall values obtained by the measurement methods, and the graph also showed good linearity. In other words, the graph is represented in the form of a linear equation of y=1.173*x+2.124.

In addition, to verify the reliable level of the simple analysis as compared to the precise analysis, a correlation between the measured values of the devices was analyzed. In this case, statistical processing was based on a statistical package for the social science (SPSS) program. Further, the correlation between the measurement devices was analyzed using a Pearson correlation coefficient (Pearson's r).

The Pearson correlation coefficient (r) is generally used to obtain a relationship between two variables. As one of the most convenient calculations, if there is a linear relationship between two variables x and y, the Pearson correlation coefficient (r) is calculated as follows.

$$r = \frac{{S}_{xy}}{\sqrt{{S}_{xx} {S}_{yy}}}$$

$$where , {S}_{xx} = {\sum }_{i = 1}^{n} {x}_{i}^{2} - \frac{{( {\sum }_{i = 1}^{n} {x}_{i} )}^{2}}{n} , \\ {S}_{yy} = {\sum }_{i = 1}^{n} {y}_{i}^{2} - \frac{{( {\sum }_{i = 1}^{n} {y}_{i} )}^{2}}{n} , and$$

$${S}_{xy} {\sum }_{i = 1}^{n} {x}_{i} {y}_{i} - \frac{( {\sum }_{i = 1}^{n} {yx}_{i} ) ( {\sum }_{i = 1}^{n} {y}_{i} )}{n} .$$

Here, the range of the Pearson correlation coefficient (r) is −1≤r≤+1.

In this case, when r=1, it means a completely positive linear correlation between two variables x and y; when r=0, it means a completely independent relationship, i.e., no linear correlation between two variables x and y; and when r=−1, it means a completely negative linear correlation between two variables x and y. When r=0, xy=0.

In general, the Pearson correlation coefficient (r) may be interpreted as shown in the following Table 1.

TABLE 1

| Range of Pearson correlation | |
|---|---|
| coefficient (r) | Meaning |
| −1.0 ≤ r ≤ −0.7 | very strong negative (−) correlation |
| −0.7 ≤ r ≤ −0.3 | strong negative(−) correlation |
| −0.3 ≤ r ≤ −0.1 | weak negative (−) correlation |
| −0.1 ≤ r ≤ 0.1 | no correlation |
| 0.1 ≤ r ≤ 0.3 | weak positive (+) correlation |
| 0.3 ≤ r ≤ 0.7 | strong positive (+) correlation |
| 0.7 ≤ r ≤ 1.0 | very strong positive (+) correlation |

FIG. 16 shows Pearson correlation analysis results between precise and simple analysis results.

In the correlation analysis results between the precise and simple analysis results of FIG. 16, the Pearson correlation coefficient shows a very strong positive (+) correlation of 0.978.

A significance probability (p-value) is 0.000, which may be interpreted as supporting a high correlation between the precise analysis and the simple analysis.

Consequently, the simple analysis has a suitably reliable level due to a high correlation with the precise analysis.

Thus, the correction for the results of the simple analysis as compared to the precise analysis is reflected to an algorithm for quantifying the furan concentration as the apparatus for diagnosing the transformer deterioration measures the chromaticity of the extraction solution. With this, the furan concentration quantified by the simple analysis is corrected to have a reliable level corresponding to that of the precise analysis.

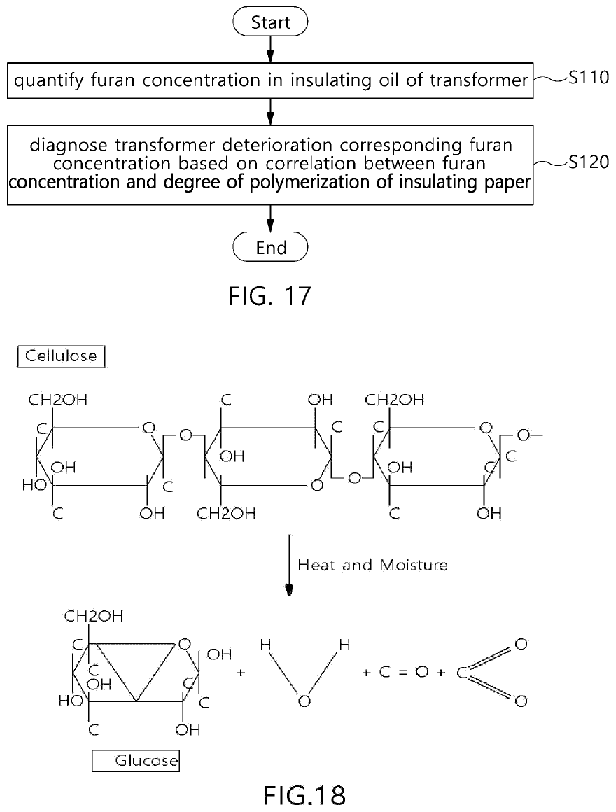

Below, the method of diagnosing the transformer deterioration will be described in detail with reference to FIG. 17.

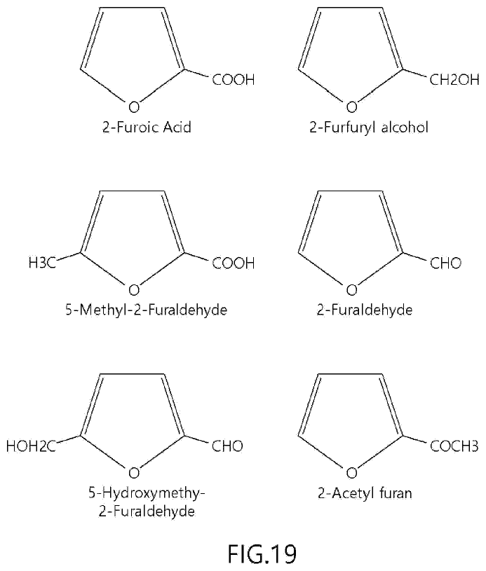

FIG. 17 shows a method of diagnosing transformer deterioration according to an embodiment of the disclosure, FIG. 18 shows a deterioration mechanism of cellulose molecules, and FIG. 19 shows a furan-based compound.

As shown in FIG. 17, the apparatus for diagnosing the transformer deterioration quantifies furan concentration in the insulating oil of the transformer (S110). In this case, the apparatus for diagnosing the transformer deterioration quantifies the furan concentration by the method of quantifying the furan concentration described above with reference to FIG. 1. Repetitive detailed descriptions about the method of quantifying the furan concentration will be avoided.

Then, the apparatus for diagnosing the transformer deterioration diagnoses the transformer deterioration corresponding to the furan concentration based on the correlation between the furan concentration and a degree of polymerization of the insulating paper (S120). In other words, the diagnostic algorithm is generated based on the correlation between the furan concentration and the degree of polymerization of the insulating paper.

Prior to description about the correlation between the furan concentration and the degree of polymerization of the insulating paper, the cellulose molecule deterioration mechanism and the furan-based compound will be described with reference to FIGS. 18 and 19.

Referring to FIG. 18, the insulating paper used as a main insulating material (a solid insulating material) of the transformer has a cellulose fiber structure extracted from a raw material of wood pulp.

Such a cellulose fiber structure includes a bunch of cellulose molecules different in length, and is structured in the form of a molecular bond based on hydroxyl (OH) and carbon (C). Cellulose itself consists of a linear polymer of glucose molecular weight and is bounded with a glycosidic molecular band.

Such a deterioration mechanism of cellulose molecules is complicated and depends on environmental conditions in use. However, according to the deterioration mechanism of cellulose molecules used as an insulating material for an electric device, it is known that deterioration caused by thermal factors is the most remarkable.

Due to the deterioration caused by moisture and heat, the glucose bond of cellulose is broken and glucose decomposition products remain in the insulating paper. Glucose produced under the conditions of moisture and acid is decomposed again to produce furfural derivatives. According to the conditions, the furfural derivatives form six structures of the furan-based compound as shown in FIG. 19.

Such furan-based derivatives provide accurate information about the decomposition of the insulating paper, and are thus used as one of diagnostic factors for the transformer deterioration. In particular, the concentration of furfural, i.e., the furan concentration may be used in indirectly evaluating the decomposition of cellulose.

Therefore, the diagnosis of the transformer based on the furan concentration is to ultimately identify how much the insulating material of the transformer has been decomposed, and prevent a trouble caused by an electric breakdown. The analysis of the correlation between the furan concentration and the degree of polymerization of the insulating paper makes it possible to diagnose the current deterioration state of the transformer because the life of the transformer is consistent with the life of the insulating material.

Thus, to derive the diagnostic algorithm for diagnosing the deterioration state of the transformer, the actual correlation between the degree of polymerization of the insulating paper and the furan concentration was analyzed.

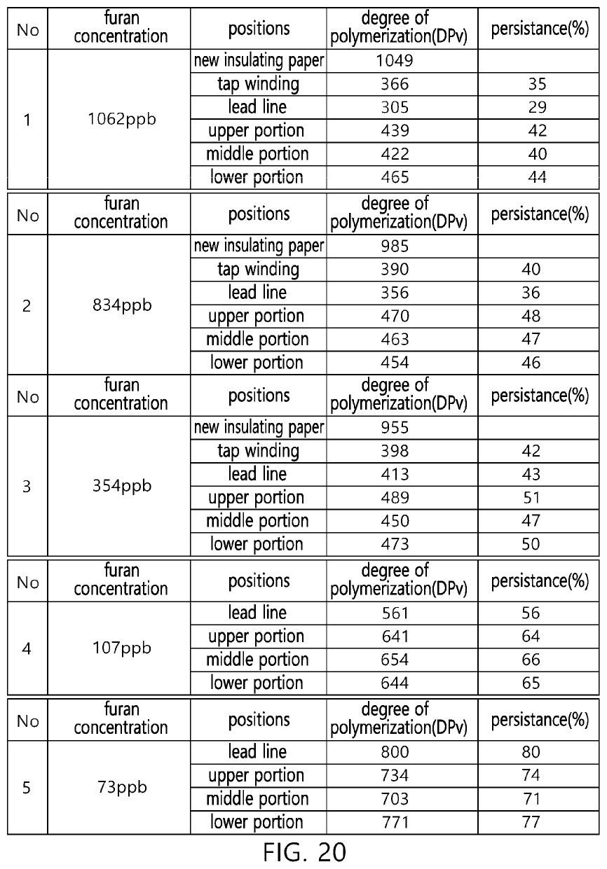

FIG. 20 shows analysis results of the degree of polymerization of the insulating paper in ground transformers.

In FIG. 20, the analysis results of the degree of polymerization of the insulating paper in the pad-mounted transformer show analysis results of a degree of polymerization of the insulating paper taken in the field, and percentages of new paper and a degree of polymerization of each insulating paper are tabulated. In addition, the new paper of the transformers No. 4 and 5 is not available from their manufacturers, and therefore an average degree of polymerization of the new paper from the other three transformers was regarded as those of the new insulating paper of the two distribution lines and converted into percentages.

Referring to FIG. 20, the higher the furan concentration, the lower the persistence of the degree of polymerization of the insulating paper. In particular, the degree of polymerization of the insulating paper around a lead line shows the lowest persistance. With this, it will be appreciated that the deterioration of the insulating paper is more serious as the furan concentration becomes higher.

In general, when the degree of polymerization is lowered to 50% or less of its initial value, it is identified that the life of the insulating paper has reached its limit. In FIG. 20, it is identified that the lives of both the transformers, in which the furan concentrations of the insulating paper are 1062 ppb and 834 ppb, have reached their limits.

In the case of the distribution transformer having a furan concentration of 354 ppb, the life of the insulating paper at all positions except upper insulating paper has reached its limit, and the life of the upper insulating paper has also reached its limit because the degree of polymerization has a persistance of 51%.

In the case of the distribution transformer having a furan concentration of 107 ppb, it may be identified that the insulating paper is good because the insulating paper around the lead line has a persistance of 56% but the insulating paper at other positions has a persistance of 60% or higher.

In the case of the distribution transformer having a furan concentration of 87 ppb, it may be identified that the insulating paper is very good because the degree of polymerization of the whole insulating paper has a persistance of 70% or higher.

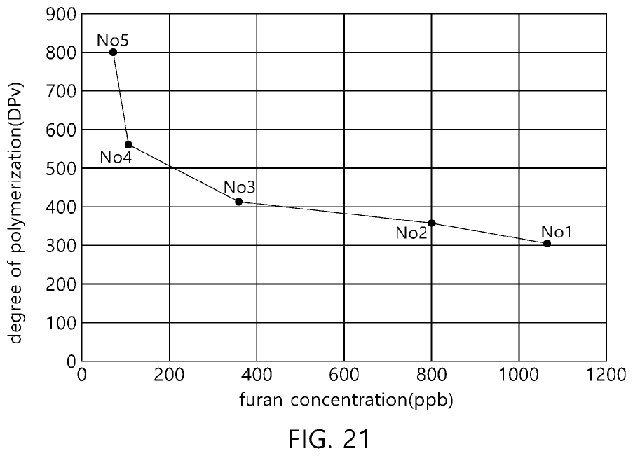

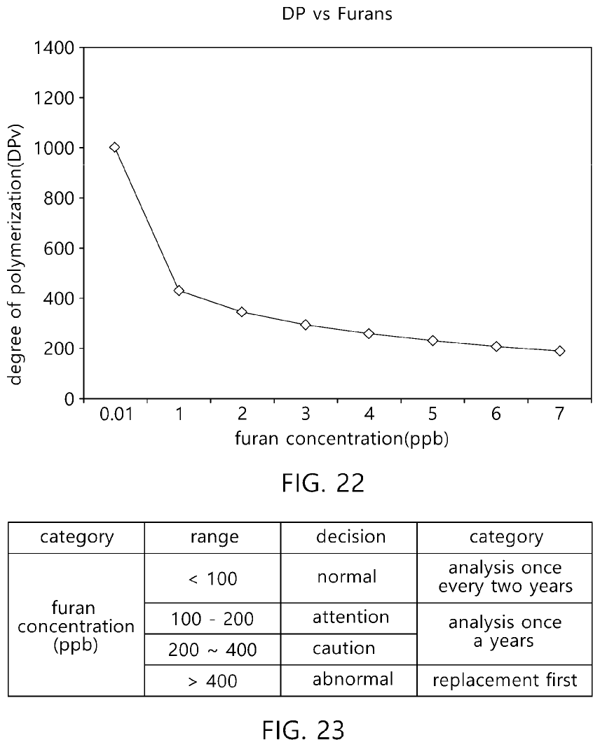

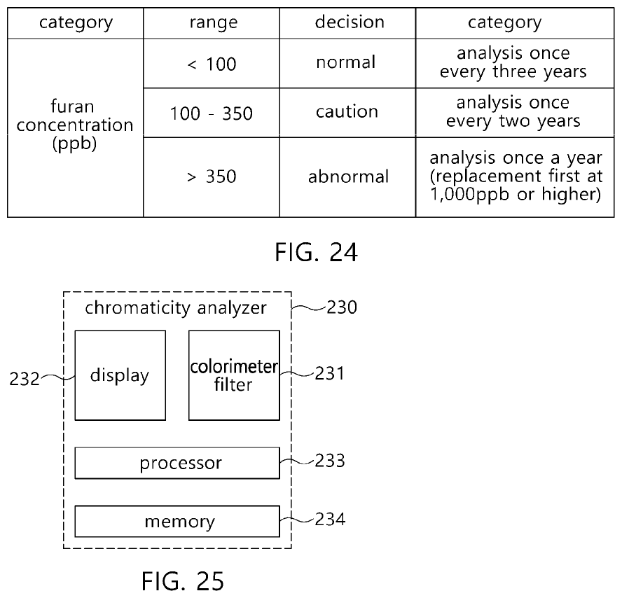

FIG. 21 shows a relationship between furan concentration of an actual onsite sample and a degree of polymerization of the insulating paper in FIG. 20, FIG. 22 shows a relationship between furan concentration and an average degree of polymerization of the insulating paper, FIG. 23 shows furan diagnostic criteria for a distribution transformer, and FIG. 24 shows furan diagnostic criteria for a transmission and transformation transformer.

FIG. 21 shows a correlation between the furan concentration and the degree of polymerization of the insulating paper around the lead line (i.e., a position where the deterioration is the most serious) in the five distribution transformers of FIG. 20.

Referring to FIG. 21, it is known that the furan concentration becomes higher as the degree of polymerization of the insulating paper decreases. It will be understood that there is a correlation between the furan concentration and the degree of polymerization of the insulating paper, and it is possible to indirectly diagnose the deterioration of the insulating material as compared to the furan concentration.

The relationship between the furan concentration of the actual onsite sample and the degree of polymerization of the insulating paper is almost similar to the relationship between the furan concentration and the average degree of polymerization of the insulating paper. The Chendong model shown in FIG. 22 shows a prediction curve of the degree of polymerization of the insulating paper versus the furan concentration.

The Chendong model refers to a model for predicting the degree of polymerization of the insulating paper from the furan concentration, in which a model equation is defined by the following Equation 1.

log₁₀(2FAL)=1.51−0.0035×DP [Equation 1]

where, 2FAL indicates the furan concentration in the insulating oil and is represented in units of mg/L, and DP indicates the average degree of polymerization of the insulating paper.

Based on the Equation 1, the deterioration state of the insulating material may be indirectly diagnosed through simulation of a relationship between the insulating oil and the insulating paper in a laboratory.

According to an embodiment of the disclosure, the apparatus for diagnosing the transformer deterioration is designed to diagnose the current state of the transformer with respect to the furan concentration through the diagnostic algorithm based on the Equation 1.

Referring to FIG. 23, the degree of polymerization of the insulating paper is lowered to 50% or less at the furan concentration of 400 ppb, the furan diagnostic criteria for the distribution transformer in the diagnostic algorithm may provide four diagnosis results of ‘normal, attention, caution, and abnormal’ with respect to this concentration.

Referring to FIG. 24, the furan diagnostic criteria for the transmission and transformation transformer (i.e., a transformer for 154 kV or higher) in the diagnostic algorithm may provide three diagnosis results of ‘normal, caution, and abnormal’ with respect to a furan concentration of 350 ppb higher than that of the distribution transformer.

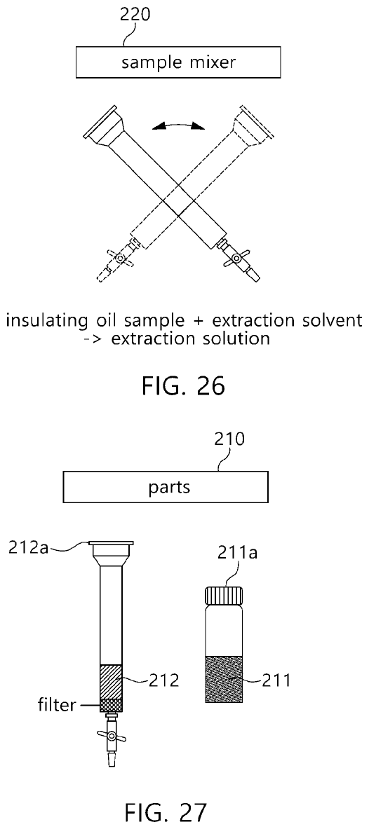

FIGS. 25 to 27 shows an apparatus for diagnosing transformer deterioration according to an embodiment of the disclosure.

As shown in FIGS. 25 to 27, an apparatus 200 for diagnosing the transformer deterioration according to an embodiment of the disclosure refers to a portable furan diagnostic apparatus that quantifies the furan compound produced by the decomposition of the insulating material in the transformer through the optimum reaction time, wavelength range and color reagent, and provides diagnosis results divided according to voltage levels.

The apparatus 200 for diagnosing the transformer deterioration includes parts 210, a sample mixer 220, and a chromaticity analyzer 230.

The parts 210 include an extraction solvent container 212a filled with an extraction solvent 212, and a color reagent container 211a filled with a color reagent 211. Here, the extraction solvent container 212a may be an econo-pac chromatography column, and the color reagent container 211a may be a vial. The extraction solvent 212 is filled in the container in consideration of the amount of reaction sample and the optimum ratio. In addition, the color reagent 211 is set to have a ratio of aniline to acetate at 1:6 based on the optimum reaction condition

Further, the sample mixer 220 regularly shakes the extraction solvent container 212a side to side at 90 degrees for mixing. In this case, the mixed solution of the extraction solvent and the insulating-oil sample is settled in the lower side of the extraction solvent container 212a so that the extraction solution can be separated as a layer.

If the extraction solvent container 212a is shaken too excessively to extract furan from the insulating-oil sample, furan is likely to be emulsified. Therefore, the sample mixer 220 is used to perform mixing at an appropriate speed. This is to extract furan from the insulating-oil sample under the optimum conditions by setting the optimum reaction time of the sample mixer 220 because an analyzer's sample mixing procedure involves high human uncertainties.

Further, the chromaticity analyzer 230 includes a display 232, a colorimeter filter 231, a processor 233, and a memory 234.

First, the display 232 is configured to display furan concentration quantified by the foregoing method of quantifying furan concentration in FIG. 1, and diagnosis results of transformer deterioration diagnosed by the method of diagnosing the transformer deterioration in FIG. 17. For example, the display 232 may be a touch screen that has an input function and a display function. In this case, the display 232 displays deterioration diagnosis results of the distribution transformer or the transmission and transformation transformer as classified according to voltage levels.

In addition, the colorimeter filter 231 is configured to measure color with which the furan concentration is quantified at the optimum wavelength range (520 nm, 530 nm) after the extraction solution reacts with the color reagent 211 and turns pink

Further, the processor 233 may also be called a controller, a microcontroller, a microprocessor, a microcomputer, etc. Further, the processor 203 may be embodied by hardware, firmware, software, or combination thereof

Further, the memory 234 may refer to a single storage device or may be a collective term for a plurality of storage elements, and is configured to store an executable program code or parameter, and data.

The memory 234 may include a random access memory (RAM), or may include a non-volatile memory (NVRAM) such as a magnetic disk storage device or a flash memory.

The processor 233 is configured to carry out the foregoing method of quantifying the furan concentration in FIG. 1, and the foregoing method of diagnosing the transformer deterioration in FIG. 17, when computer readable instructions stored in the memory 234 are executed. In other words, the apparatus 200 for diagnosing the transformer deterioration performs the foregoing method of quantifying the furan concentration in FIG. 1, and the foregoing method of diagnosing the transformer deterioration in FIG. 17

Like this, the apparatus 200 for diagnosing the transformer deterioration can accurately quantify the furan concentration and diagnose the deterioration state of the insulating material in the transformer.

As described above, the extraction solvent for the optimum reaction the mixture ratio in the color reagent, and the optimum wavelength are set to increase the accuracy in quantifying the furan concentration, the sample mixer is used to eliminate human uncertainties, and the correction algorithm for correcting the accuracy of the simple analysis as compared to the precise analysis, and the algorithm for diagnosing the transformer deterioration are included.

In addition, the apparatus 200 for diagnosing the transformer deterioration provides diagnostic results of ‘normal,’ ‘caution,’ and ‘abnormal’ by applying the diagnostic algorithm based on the furan concentration of the insulating oil in the distribution transformer and the transmission and transformation transformer, so that a user can diagnose the deterioration state of the transformer in person.

The method according to some embodiments may be implemented in the form of a program instruction executable through various computer means, and recorded in a computer readable medium. The computer readable medium may include a program instruction, a data file, a data structure, etc. independently or combination thereof. The program instruction recorded in the medium may specially be deigned or configured for the disclosure, or publicly known and usable to those skilled in the computer software art. The computer readable recording medium may for example include magnetic media such as a hard disk, a floppy disk, and a magnetic tape; optical media such a compact disk read only memory (CD-ROM), and a digital versatile disk (DVD); magneto-optical media such a floptical disk; and a ROM, RAM, a flash memory or the like hardware device specially configured to store and implement the program instruction. The program instruction may for example include not only a machine language code made by a compiler but also a high-level language code executable by a computer through an interpreter or the like.

Although the foregoing descriptions have focused on novel features according to various embodiments of the disclosure, it will be appreciated by those skilled in the art that various deletions, substitutions and changes can be made in the forms and details of the apparatuses and methods described above without departing from the scope of the disclosure. Therefore, the scope of the disclosure is defined by the appended claims rather than the foregoing description. All modifications within the scope of the equivalents of the claims are encompassed by the scope of the disclosure.

1. A furan concentration quantifying method, comprising:
measuring a color-development degree of an extraction solution by making the extraction solution containing furan extracted from an insulating-oil sample mix and react with a color reagent; and
quantifying furan concentration by correction based on a correlation between a precise analysis and a simple analysis with regard to the color-development degree of the extraction solution,
wherein the precise analysis is to obtain a quantitative value through color column separation based on high performance liquid chromatography (HPLC) in a laboratory to analyze a furan compound in insulating oil of a transformer, and
the simple analysis is to obtain a chromaticity value in a field with regard to actual transformer samples.

2. The method of claim 1, wherein the extraction solution is separated as a layer in a lower side as the insulating-oil sample and the extraction solvent are mixed and settled in a vertical direction.

3. The method of claim 2, wherein the insulating-oil sample and the extraction solvent are mixed and shaken side to side at 90 degrees for 1 minute or shaken 50 times by a sample mixer.

4. The method of claim 1, wherein a ratio of aniline to acetate is 1:6 for the color reagent.

5. The method of claim 4, wherein
a ratio of the extraction solution and the color reagent is 1:1.5, and
a reaction between the extraction solution and the color reagent is carried out for 4 minutes.

6. The method of claim 1, wherein the measuring the color-development degree of the extraction solution comprises measuring the color-development degree of the extraction solution in a wavelength range of 520 nm and 530 nm.

7. The method of claim 1, wherein the correlation between the precise analysis and the simple analysis comprises a reliable level verified through a Pearson correlation analysis based on a Pearson correlation coefficient (r) calculated by the following equation:
$$r = \frac{{S}_{xy}}{\sqrt{{S}_{xx} {S}_{yy}}} ( where , {S}_{xx} = {\sum }_{i = 1}^{n} {x}_{i}^{2} - \frac{{( {\sum }_{i = 1}^{n} {x}_{i} )}^{2}}{n} , {S}_{yy} = {\sum }_{i = 1}^{n} {y}_{i}^{2} - \frac{{( {\sum }_{i = 1}^{n} {y}_{i} )}^{2}}{n} , {S}_{xy} = {\sum }_{i = 1}^{n} {x}_{i} {y}_{i} - \frac{( {\sum }_{i = 1}^{n} {x}_{i} ) ( {\sum }_{i = 1}^{n} {y}_{i} )}{n} , \qquad [ Equation ]$$
and there is a linear relationship between two variables x and y).

8. A transformer deterioration diagnosis method, comprising:
quantifying furan concentration of an insulating-oil sample taken from a transformer; and
diagnosing a deterioration state of the transformer through the quantified furan concentration based on a correlation between the furan concentration and a degree of polymerization of insulating paper.

9. The method of claim 8, wherein the quantifying comprises:
measuring a color-development degree of an extraction solution by making the extraction solution containing furan extracted from an insulating-oil sample mix and react with a color reagent; and
quantifying furan concentration by correction based on a correlation between a precise analysis and a simple analysis with regard to the color-development degree of the extraction solution,
wherein the precise analysis is to obtain a quantitative value through color column separation based on high performance liquid chromatography (HPLC) in a laboratory to analyze a furan compound in insulating oil of a transformer, and
the simple analysis is to obtain a chromaticity value in a field with regard to actual transformer samples.

10. The method of claim 9, wherein the extraction solution is separated as a layer in a lower side as the insulating-oil sample and the extraction solvent are mixed and settled in a vertical direction.

11. The method of claim 10, wherein the insulating-oil sample and the extraction solvent are mixed and shaken side to side at 90 degrees for 1 minute or shaken 50 times by a sample mixer.

12. The method of claim 9, wherein a ratio of aniline to acetate is 1:6 for the color reagent.

13. The method of claim 12, wherein
a ratio of the extraction solution and the color reagent is 1:1.5, and
a reaction between the extraction solution and the color reagent is carried out for 4 minutes.

14. The method of claim 9, wherein the measuring the color-development degree of the extraction solution comprises measuring the color-development degree of the extraction solution in a wavelength range of 520 nm and 530 nm.

15. The method of claim 9, wherein the correlation between the precise analysis and the simple analysis comprises a reliable level verified through a Pearson correlation analysis based on a Pearson correlation coefficient (r) calculated by the following equation:
$$r = \frac{{S}_{xy}}{\sqrt{{S}_{xx} {S}_{yy}}} ( where , {S}_{xx} = {\sum }_{i = 1}^{n} {x}_{i}^{2} - \frac{{( {\sum }_{i = 1}^{n} {x}_{i} )}^{2}}{n} , {S}_{yy} = {\sum }_{i = 1}^{n} {y}_{i}^{2} - \frac{{( {\sum }_{i = 1}^{n} {y}_{i} )}^{2}}{n} , {S}_{xy} = {\sum }_{i = 1}^{n} {x}_{i} {y}_{i} - \frac{( {\sum }_{i = 1}^{n} {x}_{i} ) ( {\sum }_{i = 1}^{n} {y}_{i} )}{n} , \qquad [ Equation ]$$
and there is a linear relationship between two variables x and y).

16. The method of claim 8, wherein the diagnosing comprises diagnosing the deterioration state of the transformer with respect to furan concentration through a Chendong model showing a correlation between the furan concentration and the degree of polymerization of the insulating paper.

17. The method of claim 16, wherein the diagnosing comprises providing stepwise diagnosis results with respect to furan concentration at which the degree of polymerization of the insulating paper is lowered to 50% or less.

18. The method of claim 17, wherein the diagnosis results are providable by each of a distribution transformer and a transmission and transformation transformer.

19. An apparatus for diagnosing transformer deterioration, comprising:
at least one processor; and
a memory configured to store computer readable instructions,
wherein, the instructions are executed by the at least one processor allow the apparatus for diagnosing the transformer deterioration to:
quantify furan concentration of an insulating-oil sample taken from a transformer; and
diagnose a deterioration state of the transformer through the quantified furan concentration based on a correlation between the furan concentration and a degree of polymerization of insulating paper.

20. The apparatus of claim 19, wherein the instructions are executed by the at least one processor allow the apparatus for diagnosing the transformer deterioration to:
measure a color-development degree of an extraction solution by making the extraction solution containing furan extracted from an insulating-oil sample mix and react with a color reagent, when furan concentration is quantified; and
quantify furan concentration by correction based on a correlation between a precise analysis and a simple analysis with regard to the color-development degree of the extraction solution,
wherein the precise analysis is to obtain a quantitative value through color column separation based on high performance liquid chromatography (HPLC) in a laboratory to analyze a furan compound in insulating oil of a transformer, and
the simple analysis is to obtain a chromaticity value in a field with regard to actual transformer samples.

21. The apparatus of claim 20, wherein
the extraction solution is separated as a layer in a lower side as the insulating-oil sample and the extraction solvent are mixed and settled in a vertical direction, and
the apparatus further comprises a sample mixer configured to mix and shake the insulating-oil sample and the extraction solvent side to side at 90 degrees for 1 minute or 50 times.

22. The apparatus of claim 21, further comprising a display configured to display the quantified furan concentration, and stepwise diagnosis results with respect to the quantified furan concentration.
